SEQUENCE LISTING (7 sequences)

SEQ ID NO: 1 - DNA, 52 bp, Artificial
  Fd primer
ggggacaagtttgtacaaaaaagcaggctctagagatgagaatctggaaccg

SEQ ID NO: 2 - DNA, 56 bp, Artificial
  Rv primer
ggggaccactttgtacaagaaagctgggtcttaacggcataaagtaaggttgacat

SEQ ID NO: 3 - DNA, 19 bp, Artificial
  PDZ-GEF2 285
ggatccaacttatatagaa

SEQ ID NO: 4 - DNA, 19 bp, Artificial
  PDZ-GEF2 289
gacacggattgtattatta

SEQ ID NO: 5 - DNA, 19 bp, Artificial
  *Epac1(1)
gcacctacgtctgcaacaa

SEQ ID NO: 6 - DNA, 19 bp, Artificial
  *Epac1(2)
ccatcatcctgcgagaaga

SEQ ID NO: 7 - protein, 1601 aa, Homo sapiens
MNSPVDPGARQALRKKPPERTPEDLNTIYSYLHGMEILSNLREHQLRLMSARARYERYSGNQVLFCSETIARCWYILLSGSVLVKGSMVLPPCSFGKQFGGKRGCDCLVLEPSEMIVVENAKDNEDSILQREIPARQSRRRFRKINYKGERQTITDDVEVNSYLSLPADLTKMHLTENPHPQVTHVSSSQSGCSIASDSGSSSLSDIYQATESEVGDVDLTRLPEGPVDSEDDEEEDEEIDRTDPLQGRDLVRECLEKEPADKTDDDIEQLLEFMHQLPAFANMTMSVRRELCSVMIFEVVEQAGAIILEDGQELDSWYVILNGTVEISHPDGKVENLFMGNSFGITPTLDKQYMHGIVRTKVDDCQFVCIAQQDYWRILNHVEKNTHKVEEEGEIVMVHEHRELDRSGTRKGHIVIKATPERLIMHLIEEHSIVDPTYIEDFLLTYRTFLESPLDVGIKLLEWFKIDSLRDKVTRIVLLWVNNHFNDFEGDPAMTRFLEEFEKNLEDTKMNGHLRLLNIACAAKAKWRQVVLQKASRESPLQFSLNGGSEKGFGIFVEGVEPGSKAADSGLKRGDQIMEVNGQNFENITFMKAVEILRNNTHLALTVKTNIFVFKELLFRTEQEKSGVPHIPKIAEKKSNRHSIQHVPGDIEQTSQEKGSKKVKANTVSGGRNKIRKILDKTRFSILPPKLFSDGGLSQSQDDSIVGTRHCRHSLAIMPIPGTLSSSSPDLLQPTTSMLDFSNPSDIPDQVIRVFKVDQQSCYIIISKDTTAKEVVFHAVHEFGLTGASDTYSLCEVSVTPEGVIKQRRLPDQFSKLADRIQLNGRYYLKNNMETETLCSDEDAQELVKESQLSMLQLSTIEVATQLSMRDFDLFRNIEPTEYIDDLFKLNSKTGNTHLKRFEDIVNQETFWVASEILTEANQLKRMKIIKHFIKIALHCRECKNFNSMFAIISGLNLASVARLRGTWEKLPSKYEKHLQDLQDIFDPSRNMAKYRNILSSQSMQPPIIPLFPVVKKDMTFLHEGNDSKVDGLVNFEKLRMISKEIRQVVRMTSANMDPAMMFRQRSLSQGSTNSNMLDVQGGAHKKRARRSSLLNAKKLYEDAQMARKVKQYLSSLDVETDEEKFQMMSLQWEPAYGTLTKNLSEKRSAKSSEMSPVPMRSAGQTTKAHLHQPHRVSQVLQVPAVNLHPIRKKGQTKDPALNTSLPQKVLGTTEEISGKKHTEDTISVASSLHSSPPASPQGSPHKGYTLIPSAKSDNLSDSSHSEISSRSSIVSNCSVDSMSAALQDERCSSQALAVPESTGALEKTEHASGIGDHSQHGPGWTLLKPSLIKCLAVSSSVSNEEISQEHIIIEAADSGRGSWTSCSSSSHDNFQSLPNPKSWDFLNSYRHTHLDDPIAEVEPTDSEPYSCSKSCSRTCGQCKGSLERKSWTSSSSLSDTYEPNYGTVKRRVLESTPAESSEGLDPKDATDPVYKTVTSSTEKGLIVYCVTSPKKDDRYREPPPTPPGYLGISLADLKEGPHTHLKPPDYSVAVQRSKMMHNSLSRLPPASLSSNLVACVPSKIVTQPQRHNLQPFHPKLGDVTDADSEADENEQVSAV

(19) United States
(12) Patent Application Publication
(10) Pub. No.: US 2008/0293076 A1
(43) Pub. Date: Nov. 27, 2008

(54) Method to improve barrier function of cell-cell junctions

(75) Inventors: Johannes Lucas Bos, Bunnik (NL); Nadia Marie-Noel Dube, Utrecht (NL); Mattheus Ritske Harm Kooistra, Utrecht (NL)

Correspondence Address: TRASK BRITT, P.O. BOX 2550, SALT LAKE CITY, UT, 84110, US

(73) Assignee: UMC Utrecht Holding B.V., Utrecht (NL)

(21) Appl. No.: 12/079,688

(22) Filed: Mar. 28, 2008

(30) Foreign Application Priority Data
Apr. 4, 2007 (EP) 07105682.4

(51) Int. Cl. G01N 33/53; C12N 5/00; C12Q 1/02

(52) U.S. Cl. 435/7.2; 435/375; 435/29; 435/325

(57) ABSTRACT

Described are methods of increasing the barrier function of a cell-cell junction, comprising activating a PDZ-GEF2 protein, wherein the PDZ-GEF2 protein comprises an amino acid sequence encoded by a gene corresponding to the RapGEF6 gene. Further described are methods for selecting an activator of PDZ-GEF2, comprising contacting a plurality of candidate compounds to at least two cells of a first cell type and at least two cells of a second cell type, both cell types capable of forming cell-cell junctions, measuring the maturation of cell-cell junctions before and after contacting the two cell types with the plurality of candidate compounds, and selecting a compound from the plurality of candidate compounds that is more capable of increasing the integrity of a cell-cell junction between at least two cells of the first cell type, as compared to its capability of increasing the integrity of a cell-cell junction between cells of the second type, wherein in the second cell type, the amount and/or activity of PDZ-GEF2 is decreased, compared to the first cell type.

### PRIORITY CLAIM

This application claims priority to European Patent Application Serial No. EP 07105682.4, filed Apr. 4, 2007, titled “A New Method to Improve Barrier Function of Cell-Cell Junctions.”

### TECHNICAL FIELD

The invention relates to the integrity of cell-cell junctions in endothelial and epithelial cells and means and methods for increasing that integrity.

### BACKGROUND

Integrity of the cell-cell junctions of epithelial and endothelial cells is important for maintaining homeostasis. Epithelial cell-cell adhesion regulates tissue morphogenesis as well as cell polarity,^{[1]} and is critical to prevent unwanted migration of cells such as metastasis. For example, the integrity of cell-cell junctions of endothelial cells prevents leakage of fluid and blood cells, including platelets into the surrounding tissue. Many pathological events induce vessel leakage or are caused by vessel leakage, for example, but not limited to, disorders such as sepsis, edema, reperfusion injury and acute respiratory stress syndrome.

In the intestinal tract, the integrity and polarization of epithelial cell layers form a barrier against unwanted invasion of parasites in the body, bacteria and viruses, and against the unwanted transgression of compounds from the intestinal tract lumen to the rest of the body. A number of intercellular adhesion molecules are required to maintain polarization of epithelial cells. This organization is achieved by specialized junctions comprising adherens junctions (AJs), desmosomes, and tight junctions (TJs). AJs occur both in epithelial and endothelial cell-cell contacts and comprise, amongst others, cadherins, which interact with cadherins of neighboring cells in a calcium-dependent manner to mediate cell-cell adhesion.^{[2]}

The Ras-like small GTPases and their guanine nucleotide exchange factors (GEFs) mediate intercellular adhesion through the regulation of cytoskeletal organization, polarity and trafficking.^{[3, 4]} Evidence suggests that, amongst others, activation of the small GTPase Rap is required to recruit E-cadherin at cell-cell contact sites.^{[5]} Moreover, genetic and cell-based experiments indicate that Rap is required for proper AJs formation.^{[6, 7]} However, the molecular mechanisms and the Rap GEFs involved in this process remain unclear. One of the Rap GEFs, PDZ-GEF2 (syn.:RapGEF6, RA-GEF2), a member of the PDZ-GEF family, is an exchange factor for Rap1 and Rap2,^{[8, 9]} but its role in cell-cell junction integrity until now was unknown.

### DISCLOSURE OF THE INVENTION

Described are screening methods for selecting compounds useful for increasing cell-cell junction formation and maturation and/or useful in the treatment of syndromes related to blood vessel leakage and decreased epithelial cell wall integrity.

PDZ-GEF2, a protein that, until now, was not considered to play a role in the integrity and/or in the maturation of cell-cell junctions, mediates the maturation of cell-cell junctions and improves barrier function. We developed cells in which PDZ-GEF2 has been decreased by the action of siRNA. A decrease or inactivation of PDZ-GEF2 leads to impaired or disrupted cell-cell contacts (FIGS. 1 and 2). By comparing these cells with cells with an expression of PDZ-GEF2 higher than that of the cells with decreased PDZ-GEF2, one can proceed to the selection of compounds that induce the activation of PDZ-GEF2 and inhibit leakage of fluids and cells through blood vessel walls and/or increase the integrity of epithelial cell-cell junctions. Other methods can be used to identify compounds that activate PDZ-GEF2.

Disclosed is that the Rap guanine nucleotide exchange factor PDZ-GEF2 mediates adherens junction maturation and cell-cell integrity. Using a yeast-two-hybrid screen, we show that PDZ-GEF2 associates with the junctional scaffold protein MAGI-3 (FIG. 3). This showing is in contrast to Rap GEF PDZ-GEF1, which has been shown to associate with the junctional scaffolding molecules MAGI-1 and MAGI-2, and to bind and co-localize with the AJs protein β-catenin.^{[10-14]}

Furthermore, disclosed is that decreasing the level of PDZ-GEF2 by PDZ-GEF2-knockdown mediated by siRNA in cells, for example, in epithelial cells, abolishes Rap activation triggered by low calcium concentration (FIG. 4). In addition, disclosed is that PDZ-GEF2 knockdown leads to an immature adherens junction phenotype in cells, for example, epithelial and endothelial cells. Using real-time measurement of electrical resistance across human umbilical cord vein cell monolayers, we show that PDZ-GEF2 depletion decreases transendothelial resistance, a measure for barrier function. This effect can be rescued with 8-pCPT-2′OMe-cAMP, an agonist of Epac, a GEF for Rap1 and Rap2, showing that activation of Rap1 and/or Rap2 by PDZ-GEF2 is a critical event in the induction of barrier function. Since PDZ-GEF2 is a protein with similar function as Epac and also increases barrier function by activation of Rap1 and/or Rap2, this result implies that activation of PDZ-GEF2 by a selective compound will result in an increased barrier function of endothelial and epithelial cells.

Disclosed is that the increase and/or activation of PDZ-GEF2 increases the integrity of cell-cell junctions of cells, for example, between endothelial and/or epithelial cells by mediating the activation of Rap.

This insight enables a new method to increase barrier function of endothelial and epithelial cells by compounds capable of increasing the activity of PDZ-GEF2. Now disclosed is a method using a compound that activates PDZ-GEF2 to increase endothelial and epithelial barrier function. This compound is identified, for instance, but not exclusively, by screening for compounds that bind to PDZ-GEF2 in vitro, by screening for compounds that induce guanine nucleotide exchange activity of PDZ-GEF2 in vitro, or by screening for activation of PDZ-GEF2 in a cell.

Provided is a method of increasing the barrier function of a cell-cell junction. The method includes the activation of a PDZ-GEF2 protein, wherein the PDZ-GEF2 protein comprises an amino acid sequence encoded by a gene corresponding to the RapGEF6 gene. Of course, the PDZ-GEF2 protein also encompasses splice variants. Splice variants are sequences that occur naturally within the cells and tissues of individuals, but differ from the original sequence through alternative splicing of pre-mRNA.

This insight also enables new methods for selecting compounds capable of increasing endothelial and epithelial cell-cell integrity, thereby decreasing transport of fluid and cells through endothelial and/or epithelial barriers. For example, disclosed now is a method using an in vitro cell culture technique in which the status and integrity of cell-cell junctions can be measured, to select an activator or agonist for PDZ-GEF2. In certain embodiments, two cell types are used, wherein in one cell type, the formation and integrity of cellular junctions is at least in part inhibited by PDZ-GEF2 down-regulation or knockdown (KD), for example, mediated by siRNA (hereafter, these cells are called “PDZ-GEF2-KD cells”). In the other cell type, PDZ-GEF2 is not or to a lesser extent down-regulated (from here, these cells are called parental cells). Subsequently, a candidate compound of a plurality of compounds is administered to the cell cultures of the two cell types and the formation and/or maturation and/or integrity of cell-cell junctions are measured. Any difference in increase in the maturation or formation of the cell-cell junctions of parental cells compared to PDZ-GEF-KD cells, after contacting the cells with the compound, indicates that the compound is capable of activating PDZ-GEF2 that is present in the cells. The compound is then selected from the plurality of compounds as an agonist of PDZ-GEF2. Therefore, disclosed are methods for selecting an activator of PDZ-GEF2, such a method comprising contacting a plurality of candidate compounds to at least two cells of a first cell type and at least two cells of a second cell type, both cell types capable of forming cell-cell junctions, measuring the maturation of cell-cell junctions before and after contacting the two cell types with the plurality of candidate compounds, and selecting a compound of the plurality of candidate compounds that is more capable of increasing the integrity of a cell-cell junction between at least two cells of the first cell type, as compared to its capability of increasing the integrity of a cell-cell junction between cells of the second type, wherein in the second cell type, the amount and/or activity of PDZ-GEF2 is decreased, compared to the first cell type.

Because the herein-described method provides an activator of PDZ-GEF2, which increases the maturation of cell-cell junctions between cells, disclosed are methods to increase barrier function of epithelial and endothelial cells by activation of PDZ-GEF by any method, including, but not exclusively, methods using chemical compounds and/or antibodies, and or peptides capable of activating PDZ-GEF2.

Furthermore provided is a cell wherein PDZ-GEF2 is at least partially down-regulated, the cell characterized by immature cell-cell junctions and/or a decreased concentration of PDZ-GEF2 compared to a natural cell of the same kind. The cell may comprise an epithelial cell or an endothelial cell. The cell is particularly suitable for testing compounds that activate PDZ-GEF2.

Therefore, provided is the use of a cell according to the invention, preferably in combination with a parental cell, for the development of an assay to select an agonist of PDZ-GEF2 from a plurality of compounds and the use of a cell according to the invention for a selection assay for a compound improving the integrity of endothelial and/or epithelial cell-cell junctions.

In certain embodiments, a compound selected according to the method of the invention as described herein does not necessarily bind directly to PDZ-GEF2. For example, if an activating effect is achieved by binding of a compound selected by a method of the invention to a substance that is capable of suppressing or preventing the activation of PDZ-GEF2, then binding as described herein results in at least partial decrease of the suppressing or activation-preventing effect of the substance and thereby in the activation of PDZ-GEF2. A compound selected according to a method of the invention and having such an effect is called hereinafter an indirect agonist. Therefore, now provided is a method as described herein, wherein the compound is an indirect agonist for PDZ-GEF2.

In certain embodiments, if activation of PDZ-GEF2 is achieved by direct binding of a compound as selected according to a method of the invention, then the compound selected according to a method of the invention and having an activating effect by direct binding PDZ-GEF2, is called hereinafter a direct agonist. Therefore, now provided is a method as described herein, wherein a compound is selected that comprises a direct agonist of PDZ-GEF2. The effect of PDZ-GEF2 on cell-cell junctions results in an increased integrity of cell-cell junctions, more particularly the integrity of adherens junctions. Therefore, now provided is a method as described herein, wherein the agonist is capable of increasing integrity of a cell-cell junction. Increased integrity of cell-cell junctions results in a decrease in permeability of an endothelial or epithelial cell wall or barrier for fluid and cells. Therefore, now provided is a method as described herein, wherein the agonist is capable of decreasing permeability of an endothelial and/or epithelial cell wall or barrier.

In certain embodiments, a compound selected by a method according to the invention as described herein binds preferably to PDZ-GEF2. Therefore, provided is a method, as described herein, wherein the compound binds to PDZ-GEF2. In certain embodiments, the compound also influences and/or binds to PDZ-GEF1 and in yet other embodiments, the capability of influencing or binding to both PDZ-GEF2 as PDZ-GEF1 enhances the effect of the compound on the integrity of endothelial and/or epithelial cell-cell junctions.

The disclosure hereof enables a skilled person to select a compound capable of binding to PDZ-GEF2 or a functional part or derivative thereof. The compound may comprise a small compound or in another embodiment, a protein or proteinaceous compound. A functional part of a compound capable of binding to PDZ-GEF2 is defined as a part that has the same kind of biological properties in kind, not necessarily in amount. A functional derivative of a compound capable of binding to PDZ-GEF2 is defined as a compound that has been altered such that the biological properties of the compound are essentially the same in kind, not necessarily in amount.

In certain embodiments, provided herewith is an agonist of PDZ-GEF2, the agonist selected by a method of the invention as described herein. An agonist as selected according to the invention is very suitable for the treatment of disorders that are related to a decreased integrity of endothelial and/or epithelial walls, such as, for example, but not limited to: vessel leakage, edema, hemorrhages and diarrhea. Therefore, provided is the use of an agonist of PDZ-GEF2 according to the invention as described herein as a medicament, and also the use of an agonist of PDZ-GEF2 according to the invention as described herein for the manufacture of a medicament for decreasing vascular leakage and/or for increasing endothelial and/or epithelial integrity. The medicament may be given by the oral route or by parenteral route for the treatment of disorders that are related to a decreased integrity of endothelial and/or epithelial walls, such as, for example: vessel leakage, edema, hemorrhages and diarrhea. The medicament may be prepared by combining an agonist according to the invention with a suitable carrier and/or diluent. The carrier or diluent preferably comprises water, saline solution or another medium or carrier that is suitable and accepted for oral or parenteral administration. The invention, therefore, discloses a pharmaceutical composition comprising an agonist of PDZ-GEF2 and a suitable carrier or diluent.

The invention is further explained by the examples, without being limited by them.

### DESCRIPTION OF THE DRAWINGS

FIG. 1: PDZ-GEF2 depletion in epithelial cells impairs adherens junction formation. Panels A and B: PDZ-GEF2 knockdown leads to immature AJs. A549 cells transfected with scrambled and PDZ-GEF2 siRNAs were replated on glass coverslips for 24 hours (Panel A) and 48 hours (Panel B) following which the cells were fixed and immunostained with anti-E-cadherin (HecD1) antibody. In both panels, cells depleted in PDZ-GEF2 using the siRNA 1 (285) or 2 (289) show immature cell-cell contacts. E-cadherin junction staining was quantified by analyzing at least 350 cells from ten different fields and scoring the number of cells that exhibited mature or immature junctions according to the ratio of pixel intensity/area of the cell-cell junction. The values are presented as the average of the number of junctions scoring immature over the total E-cadherin junction staining ± standard deviation of the mean (SE). Quantification of the PDZ-GEF2 knockdown was performed using Scion Image and normalized with the corresponding loading control. Panel C: Expression levels of junctional proteins ZO-1, E-cadherin, Occludin are not affected by the absence of PDZ-GEF2. A549 cells transfected with scrambled and PDZ-GEF2 siRNAs were replated and were left untreated or treated with 4 mM EGTA for 30 minutes and equal amount of proteins were analyzed by SDS-PAGE and immunoblot. Panel D: Activated Rap1 and Epac1 rescue adherens junctions maturation in absence of PDZ-GEF2. A549 cells were treated with scrambled and PDZ-GEF2 siRNAs overnight and transfected with HA-tagged constructs (empty vector, V12Rap or Epac1) the next day. The cells were replated on glass coverslips and fixed and immunostained 24 hours later. E-cadherin junction staining was quantified and described in Panels A and B. Averages presented in the graph are means of three independent experiments. * p<0.05, ** p<0.006. (S_EV vs 285_EV: p<0.05; 285_EV vs 285_V12Rap: p<0.006; 285_EV vs 285_Epac1: p<0.05.) t-test A, * p<0.003; B, * p<0.0009, ** p<0.00004.

FIG. 2: PDZ-GEF2 knockdown in HUVECs leads to immature adherens junction and decreases endothelial resistance. Panel A: PDZ-GEF2 knockdown leads to immature VE-cadherin-based junctions in endothelial cells. HUVEC were transfected twice with scrambled, Epac1 or PDZ-GEFs siRNA and replated on glass coverslips. The cells were fixed when they formed a monolayer and immunostained with anti-VE-cadherin antibody. Panel B: Basal electrical resistance across HUVEC monolayers. Cells were treated as described in A and replated on electrodes. ECIS was measured continuously for 15 hours and the values reported are the final time points. Panel C: 007 increases endothelial resistance in HUVEC and rescues the PDZ-GEF2 knockdown phenotype. The values reported correspond to the endothelial resistance measured 90 minutes after adding 007 to the culture media. * p<0.03 control mock vs 007; p<0.001 PDZ-GEF2.285 mock vs 007.

FIG. 3: Identification of MAGI-3 as a PDZ-GEF2-interacting protein. Panel A: Schematic representation of the full-length MAGI-3 protein and the MAGI-3 clones 4, 7, 12 and 6 identified in the yeast-two-hybrid screen of human placenta cDNA library. PDZ, PSD-95/disc large/zona occludens 1 domain; GuK, guanylate kinase domain. Panel B: Co-immunoprecipitation of HA-PDZ-GEF2 full length with V5-MAGI-3 clone 6 from 293T cells (top panel). 293T cells transfected with 3 μg of plasmid cDNAs encoding V5-MAGI-3 clone 6, HA-empty vector, HA-PDZ-GEF2 full length, HA-PDZ-GEF2 C-terminal truncated and HA-Epac1 and lysed in modified RIPA 48 hours post-transfection. V5-MAGI-3 clone 6 was immunoprecipitated using V5 antibody and protein A beads. Samples were subjected to 10% SDS-PAGE electrophoresis, followed by immunoblotting using anti-V5 and anti-HA (12CA5) antibodies. Neither HA-PDZ-GEF2 C-terminal truncated nor HA-Epac1 co-immunoprecipitate with V5-MAGI-3 clone 6 (WW-WW-PDZ). Expression of the constructs is shown on the bottom panels (TCL). The results are representative of three independent experiments.

FIG. 4: Activation of Rap1 by calcium depletion in A549 cells is dependent on PDZ-GEF2 expression. A549 cells were transfected with scrambled or PDZ-GEF2 RNAi oligos and replated 24 hours before the calcium switch at a density ensuring confluency at the time of the experiment. Cells were rinsed with PBS and treated with 4 mM EGTA for 30 minutes to disrupt Ca²⁺-dependent cell adhesion, following which the cells were lysed or rinsed with PBS and incubated in serum-free media containing 20 μM calcium. The cells were lysed at the indicated time points and the samples were incubated with immobilized GST-RalGDS-Ras binding domain to precipitate active (GTP-bound) Rap.

FIG. 5: Schematic representation of the PDZ-GEF2 protein. PDZ-GEF2 domain organization: cNBD (amino acids 280 to 398 of SEQ ID NO:______), cyclic nucleotide binding domains; RasGEFN (amino acids 412 to 525 of SEQ ID NO:______), N-terminal Ras-GEF domain; PDZ (amino acids 540 to 612 of SEQ ID NO:______), domain present in PSD-95, Dgl, and ZO-1; RA, Ras-associating domain; RasGEF (amino acids 856 to 1138 of SEQ ID NO:______), guanine-nucleotide dissociation stimulator CDC25 homology domain; xPPxY (amino acids 1507 to 1511 and 1528 to 1532 of SEQ ID NO:______), protein-protein interaction PY-motifs; and SAV (amino acids 1599 to 1601 of SEQ ID NO:______), C-terminal PDZ binding motif.

FIG. 6: Amino-acid sequence of PDZ-GEF2. Indicated is the amino acid sequence of PDZ-GEF2 (SEQ ID NO:______), a protein encoded by the RapGEF6 gene. The gene may encode splice variants of PDZ-GEF2, proteins which are included in the invention.

### DETAILED DESCRIPTION OF THE INVENTION

### Examples

### Materials and Methods

### Cell Culture

293T cells were maintained in DMEM (BioWhittaker) supplemented with 10% fetal bovine serum (FBS) (Cambrex), 1.2 mM L-Glutamine (BioWhittaker) and antibiotics (100 units/ml penicillin, 100 μg/ml streptomycin) (BioWhittaker). A549 cells were cultured in RPMI (BioWhittaker) supplemented with 10% FBS and antibiotics as described herein. HUVEC were isolated according to Jaffe et al.^{[16, 17]} and cultured on 1% gelatin (Sigma) in EBM-2 Bulletkit culture medium (EBM-2 supplemented with EGM-2 SingleQuots (HEGF, hydrocortisone, fetal bovine serum, VEGF, HFGF-B, R3-IGF-1, ascorbic acid, GA-1000, heparin) (Clonetics)). Second to fifth passage cells were used.

### Antibodies

Monoclonal antibodies 5D3 against Epac1 have been described previously.^{[7]} Additional primary antibodies were purchased from AbCam (anti-E-Cadherin HecD1), GeneTex (anti-E-Cadherin HecD1), Invitrogen (anti-V5), Oncogene (anti-alpha-tubulin), Santa Cruz Biotechnology (anti-Rap), Sigma (anti-MAGI-3), Transduction Laboratories (p130cas, β-catenin, VE-cadherin: Cadherin-5), Zymed (anti-occludin, anti-ZO-1), anti-hemagglutinin (HA) monoclonal antibody 12CA5. Anti-mouse and anti-rabbit fluorescent secondary antibodies (FITC and TRITC) were from Molecular Probes (Eugene, Oreg., US).

### PDZ-GEF2 Antibody Production

To produce antibodies reactive against PDZ-GEF2, the first nucleotide binding domain of human PDZ-GEF2 corresponding to amino acids 1-123 was subcloned into the bacterial expression vector pGEX. Glutathione S-transferase (GST) fusion protein was produced and used to immunize rabbits.

### Yeast-Two-Hybrid Screen

Full-length PDZ-GEF2 was subcloned into the pB38 vector and used as a bait in a yeast-two-hybrid system with a human placenta cDNA library. The screen was performed by Hybrigenics as described in reference 18.

### Expression Vectors and Cloning/DNA Construction

The following hemagglutinin (HA)-tagged expression vectors were described previously: pMT2-HA-PDZ-GEF2 full length and pMT2-HA-PDZ-GEF2 ΔC;^{[9]} pMT2-HA-Epac1;^{[19]} pMT2-HA-V12Rap.^{[20]} The mouse MAGI-3 cDNA clone IMAGp998A228567Q was obtained from the German Resource Center for Genome Research (RZPD) and used to clone the WW domains and the second PDZ domain of MAGI-3, which correspond to the interacting part found in the yeast-two-hybrid screen (clone 6). Amino acids 291-504 corresponding to the mouse WW-WW-PDZ domains of MAGI-3 were generated by PCR amplification and cloned into a Gateway entry vector (Invitrogen) using the following primer set:

Forward 5′-GGGGACAAGTTTGTACAAAAAAGCAGGCTCTAGAGATGAGAATCTGGAACCG-3′ (SEQ ID NO:______);

Reverse 5′-GGGGACCACTTTGTACAAGAAAGCTGGGTCTTAACGG CATAAAGTAAGGTTGACAT-3′ (SEQ ID NO:______). The MAGI-3 Y2H (clone 6) entry clone was recombined into a V5-pcDNA3 destination vector following the manufacturer's instructions. The V5-pcDNA3-MAGI-3 Y2H vector was sequenced to ensure proper identity of the amino acids.

### Transfection

293T and A549 cells were transfected using FuGENE6 reagent for 48 hours (Roche Diagnostics Corporation). For siRNA, A549 cells and HUVEC were transfected using oligofectamine (Invitrogen) for 24 to 48 hours according to the manufacturer's instructions. In the case of the HUVEC, transfection was repeated after 24 hours. Samples were analyzed 48 hours after the second round of transfection. Oligos sequences (Dharmacon): PDZ-GEF2 (285: GGATCCAACTTATATAGAA) (SEQ ID NO:______); (289: GACACGGATTGTATTATTA) (SEQ ID NO:______); *Epac1 (GCACCTA CGTCTGCAACAA) (SEQ ID NO:______) and (CCATCATCCTGCGAGAAGA) (SEQ ID NO:______). 8-pCPT-2′O-Me-cAMP/8-pCPT -2′-OMe-cAMP (Biolog) was used at a final concentration of 100 μM. Quantification of the PDZ-GEF2 knockdown was performed using Scion Image (Scion Corporation).

### Immunofluorescence

A549 cells transfected with DNA expression vectors and/or siRNA oligos were plated on coverslips. Twenty-four or 48 hours later, cells were rinsed twice with ice cold PBS and fixed in a solution of 3.8% formaldehyde, permeabilized in 0.1% Triton X-100 and blocked with 2% BSA in PBS at room temperature for one hour or overnight at 4° C. The coverslips were incubated with the indicated primary antibodies for one hour followed by 30 minutes incubation with the Alexa secondary antibodies, then mounted on glass slides and dried overnight. The cells were examined using a Zeiss Axiokop2 microscope or by confocal microscopy. Pictures were taken at 40× magnification and were arranged and resized using Adobe Photoshop CS. The values are presented as the average of the number of junctions scoring immature over the total E-cadherin junction staining ± standard deviation of the mean (SE).

### Calcium Switch Experiments

A549 cells transfected with scrambled or PDZ-GEF2.285 oligos were replated in growth media 24 hours before calcium switch. The dishes were then rinsed twice with PBS and serum-free and calcium-free media containing 4 mM EGTA was added to the cells for 30 minutes at 37° C. to disrupt E-cadherin-mediated cell-cell contacts. Following calcium chelation, cells were lysed or incubated in media supplemented with low calcium (20 μM) for 20 and 60 minutes.

### Immunoprecipitation and Western Blotting

Cells were washed in ice-cold PBS and lysed in modified RIPA buffer (150 mM NaCl, 50 mM Tris-HCl pH 7.4, 1% Nonidet P-40) containing protease inhibitors, sodium vanadate and sodium fluoride. Cell lysates were cleared by centrifugation and rotated with anti-V5 or anti-HA antibodies and protein A-Agarose at 4° C. for two hours. Precipitates were washed four times in lysis buffer, resuspended in SDS sample buffer, resolved by 8% SDS-PAGE and transferred to polyvinylidene difluoride membrane (PVDF; Immobilon, Millipore). The membranes were blocked in 1% bovine serum albumin (BSA) and 1% non-fat milk, followed by incubation with primary antibodies overnight at 4° C. Horseradish peroxidase-conjugated anti-mouse or anti-rabbit was used as a secondary antibody and the blot was developed using enhanced chemiluminescence.

### Endothelial Permeability Assay

To assess endothelial permeability, we measured electrical resistance continuously across endothelial cell monolayer using the methodology known as “Electric Cell-Substrate Impedance Sensing” (ECIS).^{[21]} Briefly, HUVEC were grown to confluence on small gold electrodes and ECIS was subsequently monitored for up to 15 hours. Data was analyzed using the Applied Biophysics software.

### Results

### The Impact of PDZ-GEF2 Knockdown on Junction Formation

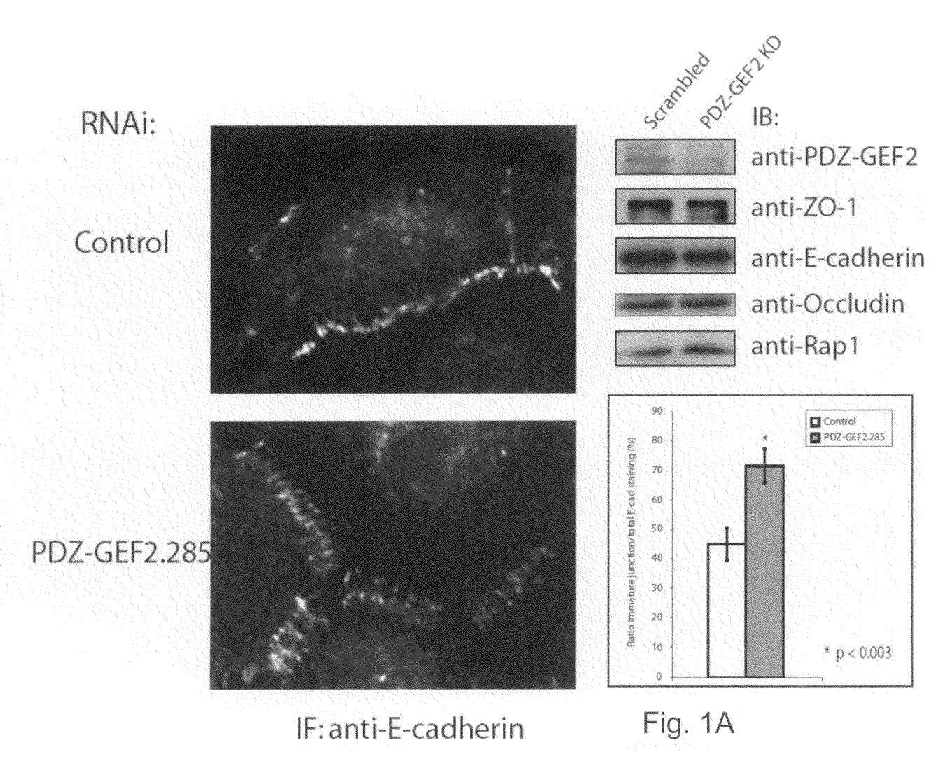

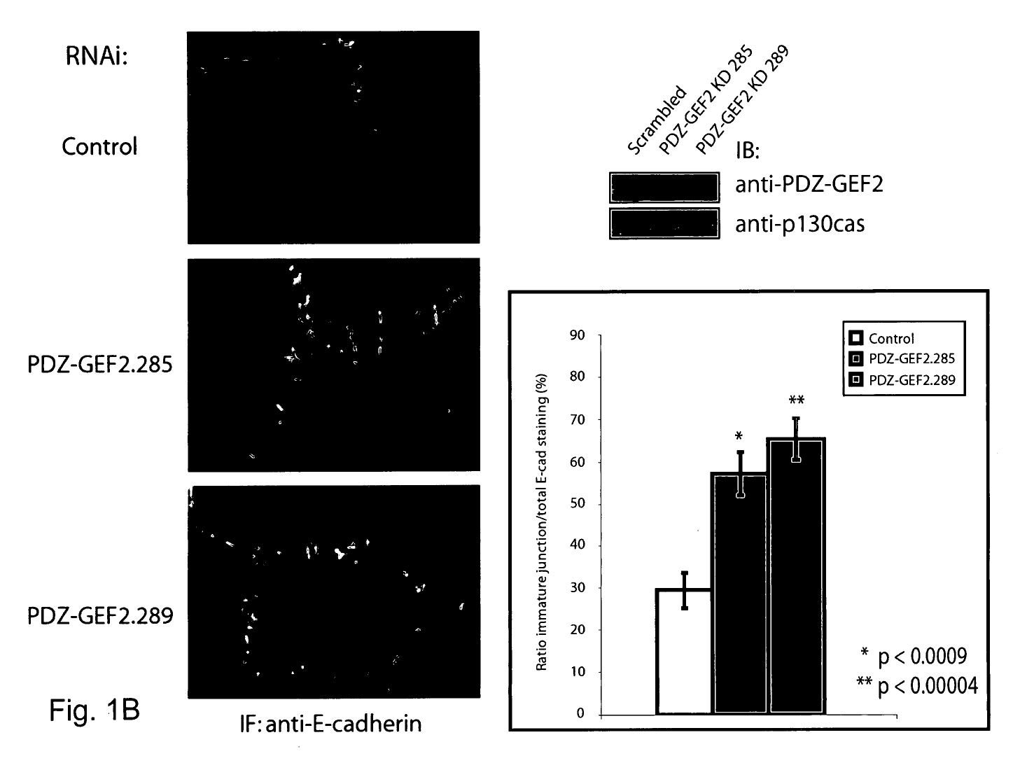

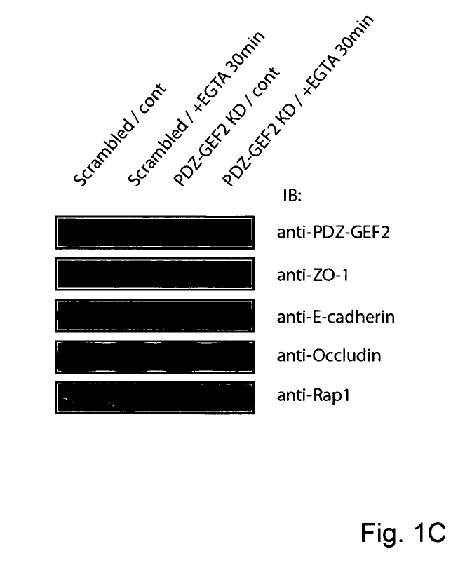

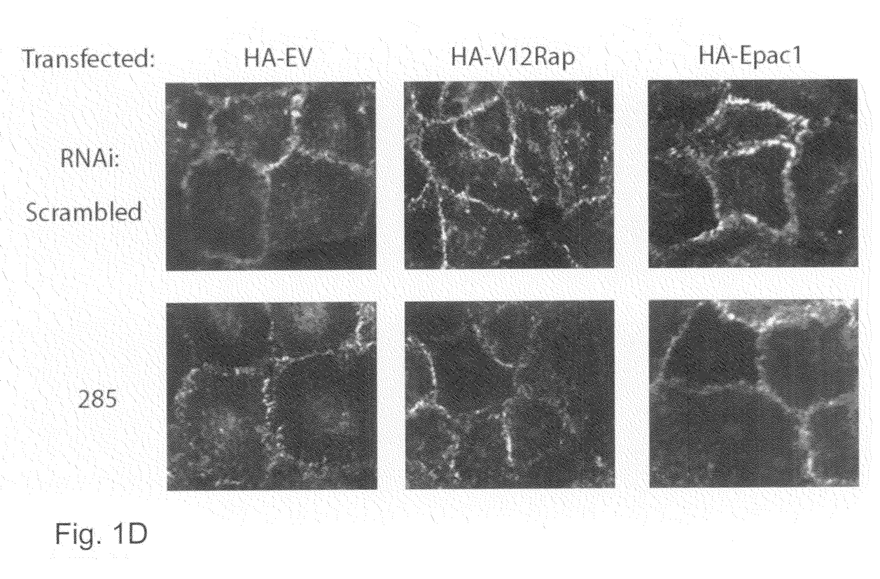

In epithelial cells, the presence of E-cadherin protein mediates the establishment of cell-cell contacts (reviewed in reference 22). The presence of E-cadherin in mature cell-cell contacts can be visualized in vitro. Therefore, PDZ-GEF2-depleted A549 cells were replated on glass coverslips and E-cadherin staining was performed. Treatment with PDZ-GEF2 siRNAi for 48 hours effectively reduced the levels of endogenous PDZ-GEF2 protein to 64% and 52% of baseline (FIG. 1, Panels A and B, oligos 285 and 289, respectively). Remarkably, PDZ-GEF2 depletion was associated with suppression of cell-cell contacts maturation 24 and 48 hours following seeding of the cells (FIG. 1, Panels A and B, respectively). The junctions depicted are zipper-like structures, as seen in epithelial cells establishing initial cell-cell contacts.^{[23]} We quantified the amount of these immature junctions and obtained approximately twice as much in the absence of PDZ-GEF2 (graphs of FIG. 1, Panels A and B). A similar pattern of localization was observed for β-catenin, another marker of adherens junction integrity (data not shown). To exclude impairment of E-cadherin or other junctional proteins expression in absence of PDZ-GEF2, we examined the levels of E-cadherin, ZO-1 and occludin in cells treated or not treated with 4 mM EGTA for 30 minutes and found equal amounts in scrambled and PDZ-GEF2 siRNA-treated cells (FIG. 1, Panels A and C). Interestingly, the PDZ-GEF2 knockdown phenotype (PDZ-GEF2-KD) is similar to the one observed in Rap1A-depleted cells (data not shown), providing evidence that PDZ-GEF2 is required for Rap activation and further supporting a role for Rap in junction establishment.

### Expression of Activated Rap or Epac1 Rescues Adherens Junction Maturation in PDZ-GEF2-Depleted Cells (PDZ-GEF2-KD)

To confirm that the immature junction phenotype was caused by PDZ-GEF2 depletion that down-regulated Rap activation, activated Rap (Vl2Rap) and Epac1 were re-expressed in scrambled or PDZ-GEF2 siRNA-transfected cells and anti-E-cadherin staining was performed. Expression of these constructs is known to lead to Rap activation and significantly restored junction maturation to levels observed in cells transfected with the scrambled oligo and empty vector (FIG. 1, Panel D). We concluded that Rap activation by PDZ-GEF2 is critical for cell-cell contact maturation in A549 cells.

### Increased Endothelial Permeability in HUVEC Depleted in PDZ-GEF2 (PDZ-GEF2-KD)

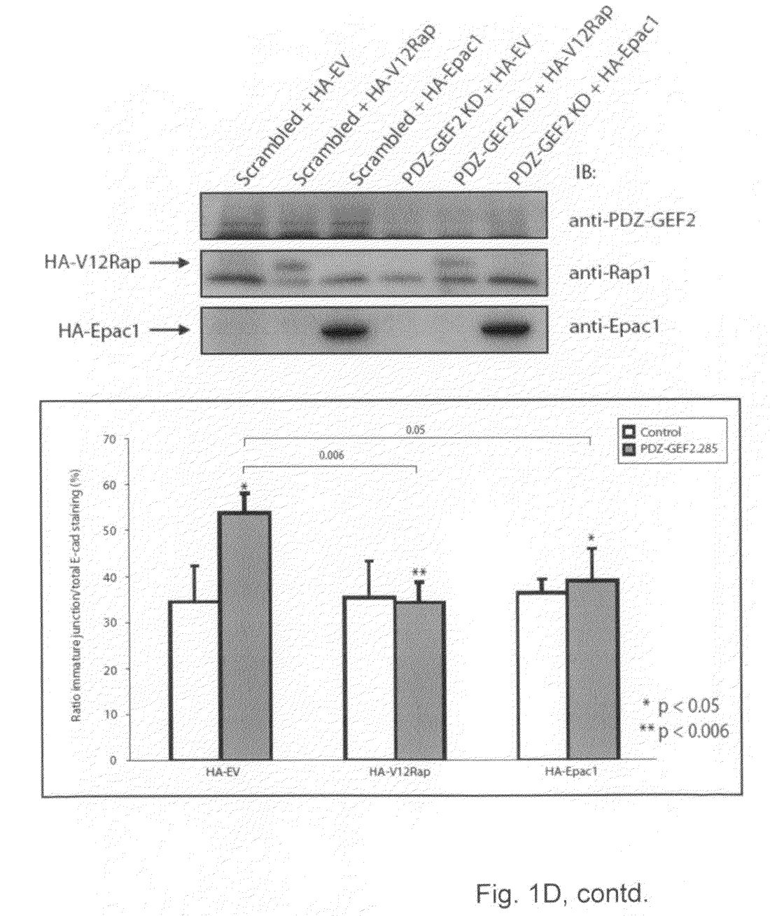

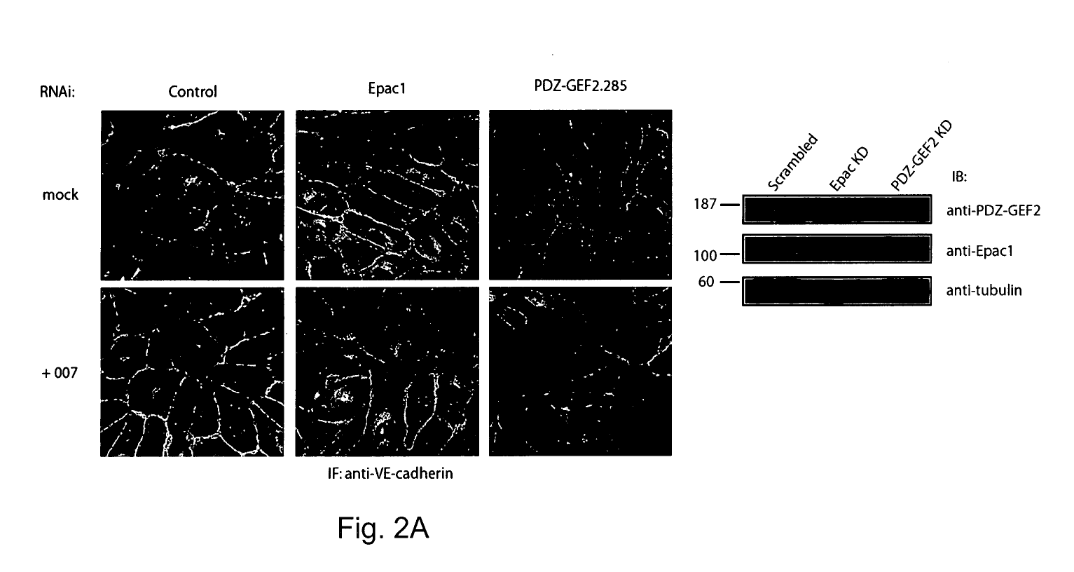

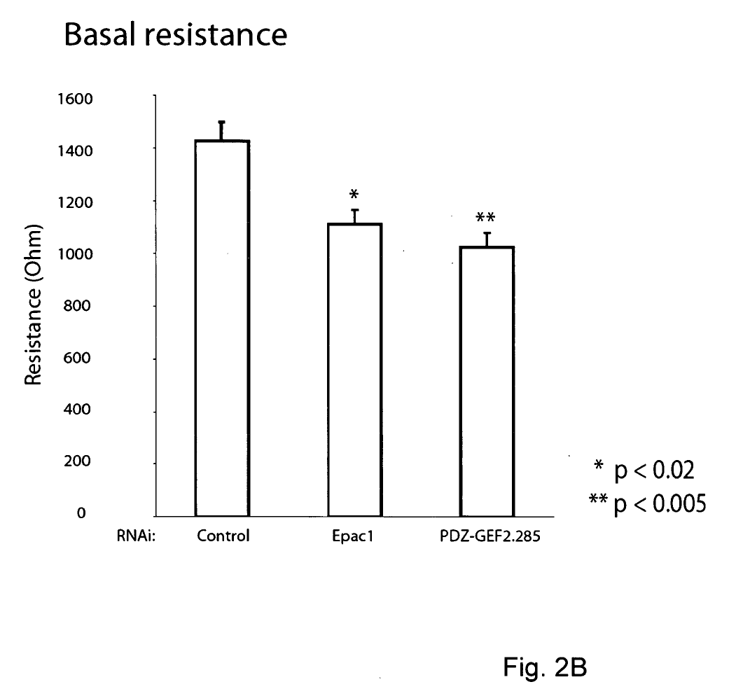

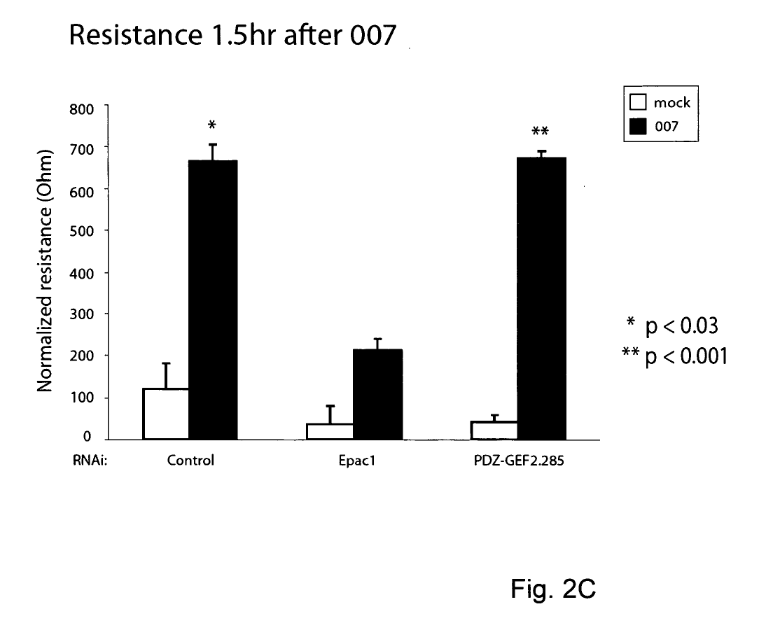

We next showed the role of PDZ-GEF2 for adherens junction formation in endothelial cells. HUVEC were transfected as described and seeded on glass coverslips or on electrodes for ECIS measurement of cellular resistance. Experiments were performed when cells formed a confluent layer. In FIG. 2, Panel A, VE-cadherin staining of PDZ-GEF2-depleted HUVEC (PDZ-GEF2-KD) clearly showed zipper-like, immature junctions, whereas scrambled siRNA had no effect on junction appearance. It is known that immature junctions correlate with increased cell permeability and that the Rap GEF Epac1 regulates this process.^{[24-27]} Therefore, we studied electrical resistance of HUVEC treated with Epac1 and PDZ-GEF2 siRNA. As previously reported, basal electrical resistance was decreased in Epac1-depleted cells (FIG. 2, Panel B). The morphological phenotype was confirmed by electrical resistance functional assay. As expected, the reduced maturation of cell-cell contacts in absence of PDZ-GEF2 correlated with a 30% decrease in basal electrical resistance (FIG. 2, Panel B). Activation of Rap through Epac1 by its specific agonist 8-pCPT-2′-OMe-cAMP is known to decrease cell permeability of endothelial cells by tightening the junctions.^{[26]} Accordingly, a 30-minute stimulation with 8-pCPT-2′-OMe-cAMP (100 μM) increased resistance in scrambled siRNA cells and the decreased resistance observed in PDZ-GEF2-depleted cells was completely rescued by 8-pCPT-2′-OMe-cAMP (FIG. 2, Panel C). As a control, HUVEC transfected with Epac1 siRNA were stimulated with 8-pCPT-2′-OMe-cAMP and a minimal effect on electrical resistance was observed. These findings indicate that PDZ-GEF2 is required to maintain endothelial cell integrity. Moreover, the impaired junction maturation was consistent with decreased electrical resistance in PDZ-GEF2-depleted HUVEC. Furthermore, activation of Epac by 8-pCPT-2′-OMe-cAMP could rescue the defect.

### Identification of MAGI-3 as a PDZ-GEF2-Interacting Protein

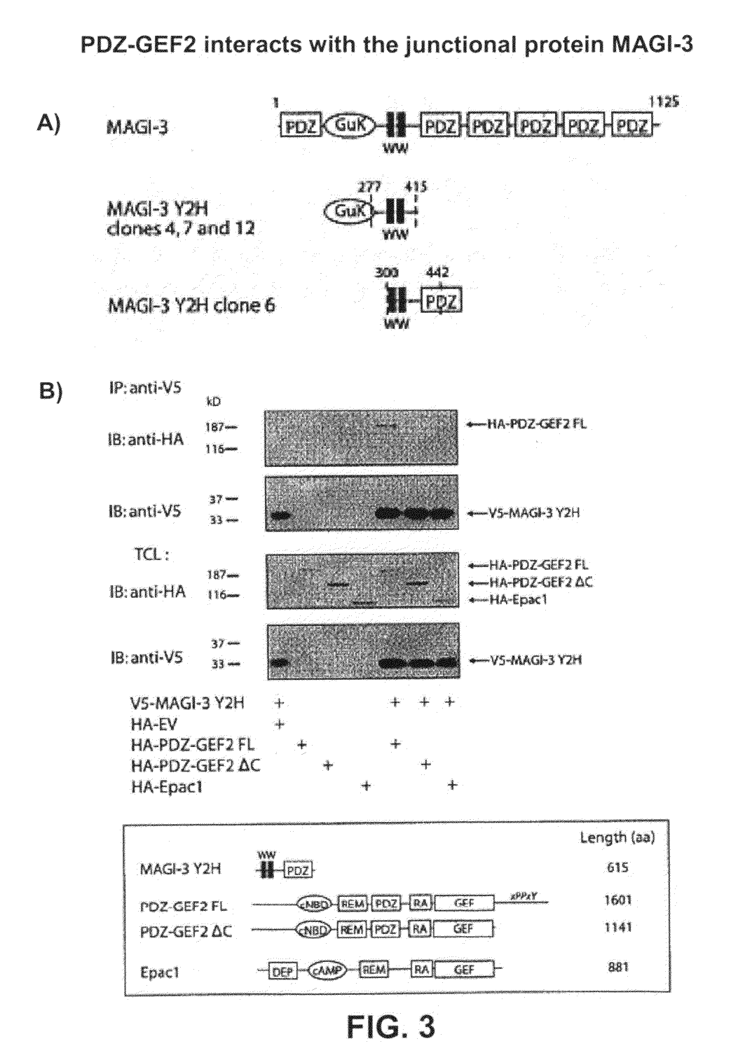

In a yeast-two-hybrid screen using PDZ-GEF-2 as a bait, we found MAGI-3 as an interaction partner of PDZ-GEF-2. The MAGI-3 protein comprises a PDZ (PSD-95/disc large/zona occludens 1) domain and a GuK (guanylate kinase) domain and is a member of the MAGI family. Furthermore, HA-PDZ-GEF2 full-length co-immunoprecipitates with V5-MAGI-3 clone 6 when co-expressed in 293T cells (FIG. 3).

### PDZ-GEF-2 Required for Rap1 Activation

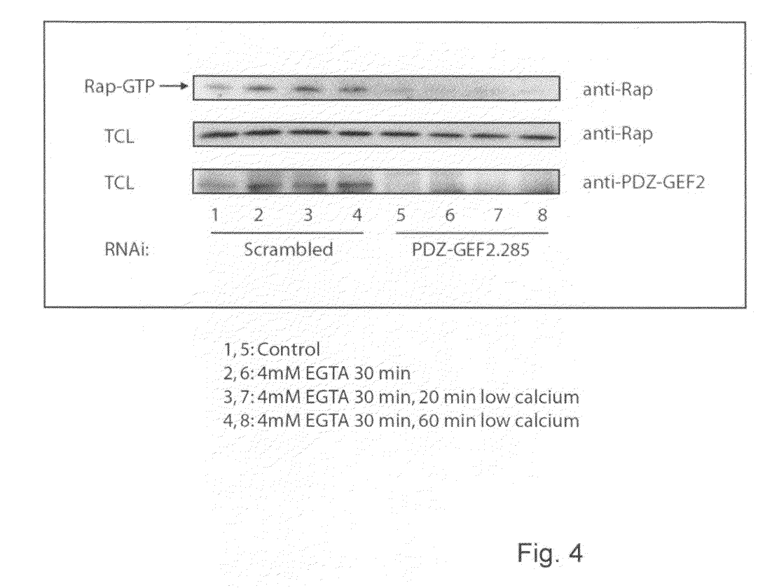

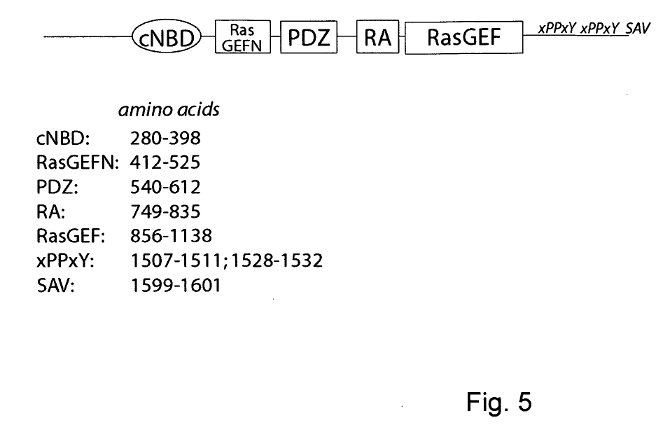

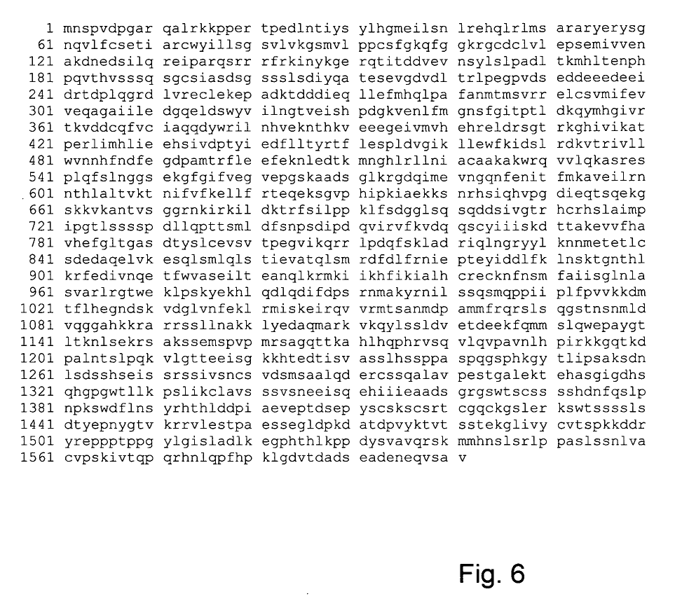

To investigate whether PDZ-GEF-2 is required for Rap activation during junction remodeling, we introduced PDZ-GEF2 siRNA in A549 cells and determined its effect on Rap-GTP levels upon disruption of cell junctions by EGTA. In control siRNA-treated cells, EGTA-induced Rap1 activation (FIG. 4, Panel A, lane 2), compatible with previous results. To exclude effects of EGTA, we have replaced the EDTA for low Ca²⁺ (20 μM). Under these conditions, junctions are not formed (data not shown) and Rap1 remains elevated (FIG. 4, Panel A, lanes 3 and 4). In PDZ-GEF2-depleted cells, EGTA treatment was ineffective in increasing Rap1-GTP levels (FIG. 4, Panel A, lanes 6-8), showing that PDZ-GEF2 is required for Rap1 activation triggered by the disruption of E-cadherin-based junctions.

### REFERENCES

### The Contents of the Entirety of Each of Which are Incorporated by this Reference)

1. Chen X. and B. M. Gumbiner, Crosstalk between different adhesion molecules. Curr. Opin. Cell Biol. 2006, 18(5):572-8.
2. Vasioukhin V. and E. Fuchs, Actin dynamics and cell-cell adhesion in epithelia. Curr. Opin. Cell Biol. 2001, 13(1):76-84.
3. Bos J. L., Linking Rap to cell adhesion. Curr. Opin. Cell Biol. 2005, 17(2):123-8.
4. Braga V. M. and A. S. Yap, The challenges of abundance: epithelial junctions and small GTPase signaling. Curr. Opin. Cell Biol. 2005, 17(5):466-74.
5. Hogan C. et al., Rapt regulates the formation of E-cadherin-based cell-cell contacts. Mol. Cell Biol. 2004, 24(15):6690-700.
6. Knox A. L. and N. H. Brown, Rap1 GTPase regulation of adherens junction positioning and cell adhesion. Science 2002, 295(5558):1285-8.
7. Price L. S. et al., Rapt regulates E-cadherin-mediated cell-cell adhesion. J. Biol. Chem. 2004, 279(34):35127-32.
8. Gao X. et al., Identification and characterization of RA-GEF-2, a Rap guanine nucleotide exchange factor that serves as a downstream target of M-Ras. J. Biol. Chem. 2001, 276(45):42219-25.
9. Kuiperij H. B. et al., Characterization of PDZ-GEFs, a family of guanine nucleotide exchange factors specific for Rap1 and Rap2. Biochim. Biophys. Acta. 2003, 1593(2-3): 141-9.
10. Dobrosotskaya I. Y. and G. L. James, MAGI-1 interacts with beta-catenin and is associated with cell-cell adhesion structures. Biochem. Biophys. Res. Commun. 2000, 270(3):903-9.
11. Kawajiri A. et al., Identification of a novel beta-catenin-interacting protein. Biochem. Biophys. Res. Commun. 2000, 273(2):712-7.
12. Mino A. et al., Membrane-associated guanylate kinase with inverted orientation (MAGI)-1/brain angiogenesis inhibitor 1-associated protein (BAP1) as a scaffolding molecule for Rap small G protein GDP/GTP exchange protein at tight junctions. Genes Cells 2000 5(12):1009-16.
13. Ohtsuka T. et al., nRap GEP: a novel neural GDP/GTP exchange protein for rapI small G protein that interacts with synaptic scaffolding molecule (S-SCAM). Biochem. Biophys. Res. Commun. 1999, 265(1):38-44.
14. Sakurai A. et al., MAGI-1 is required for Rap1 activation upon cell-cell contact and for enhancement of vascular endothelial cadherin-mediated cell adhesion. Mol. Biol. Cell 2006, 17(2):966-76.
15. Arndt K. and K. Muller, Protein engineering protocols. Methods in Molecular Biology 2007, Vol. 352:95-109.
16. Jaffe E. A. et al., Culture of human endothelial cells derived from umbilical veins. Identification by morphologic and immunologic criteria. J. Clin. Invest. 1973, 52(11):2745-56.
17. Willems C. et al., Media conditioned by cultured human vascular endothelial cells inhibit the growth of vascular smooth muscle cells. Exp. Cell Res. 1982, 139(1): p. 191-7.
18. Rain J. C. et al., The protein-protein interaction map of Helicobacter pylori. Nature 2001, 409(6817):211-5.
19. de Rooij J. et al., PDZ-GEF1, a guanine nucleotide exchange factor specific for Rap1 and Rap2. J. Biol. Chem. 1999, 274(53):38125-30.
20. Zwartkruis F. J. et al., Extracellular signal-regulated activation of Rap1 fails to interfere in Ras effector signaling. Embo. J. 1998, 17(20):5905-12.
21. Tiruppathi C. et al., Electrical method for detection of endothelial cell shape change in real time: assessment of endothelial barrier function. Proc. Natl. Acad. Sci. U.S.A. 1992, 89(17):7919-23.
22. Lien W. H., O. Klezovitch, and V. Vasioukhin, Cadherin-catenin proteins in vertebrate development. Curr. Opin. Cell Biol. 2006, 18(5):499-506.
23. Vasioukhin V. et al., Directed actin polymerization is the driving force for epithelial-cell-cell adhesion. Cell 2000, 100(2):209-19.
24. Cullere X. et al., Regulation of vascular endothelial barrier function by Epac, a cAMP-activated exchange factor for Rap GTPase. Blood 2005, 105(5):1950-5.
25. Fukuhara S. et al., Cyclic AMP potentiates vascular endothelial cadherin-mediated cell-cell contact to enhance endothelial barrier function through an Epac-Rap1 signaling pathway. Mol. Cell Biol. 2005, 25(1):136-46.
26. Kooistra M. R. et al., Epac1 regulates integrity of endothelial cell junctions through VE-cadherin. FEBS Lett. 2005, 579(22):4966-72.
27. Wittchen E. S. et al., Rap1 GTPase inhibits leukocyte transmigration by promoting endothelial barrier function. J. Biol. Chem. 2005, 280(12):11675-82.

1. A method of increasing the barrier function of a cell-cell junction, the method comprising:
activating a PDZ-GEF2 protein.

2. The method according to claim 1, wherein activating a PDZ-GEF2 protein comprises the use of an agonist that is an indirect agonist of PDZ-GEF2.

3. The method according to claim 1, wherein activating a PDZ-GEF2 protein comprises the use of an agonist that is a direct agonist of PDZ-GEF2.

4. The method according to claim 2, wherein the agonist increases integrity of the cell-cell junction.

5. The method according to claim 2, wherein the agonist decreases permeability of an endothelial and/or epithelial cell layer.

6. The method according to claim 2, wherein the agonist binds to PDZ-GEF2.

7. A method for selecting an activator of PDZ-GEF2, the method comprising:
contacting a plurality of candidate compounds with at least two cells of a first cell type and at least two cells of a second cell type, both said first and second cell types capable of forming cell-cell junctions,
measuring the maturation of cell-cell junctions before and after contacting the cell types with the plurality of candidate compounds, and
selecting a compound from the plurality of candidate compounds that is more capable of increasing the integrity of a cell-cell junction between the two cells of the first cell type, as compared to its capability of increasing the integrity of a cell-cell junction between cells of the second type, wherein, in the second cell type, the amount and/or activity of PDZ-GEF2 is decreased, compared to the first cell type.

8. The method according to claim 7, where in the compound is an indirect agonist of PDZ-GEF2.

9. The method according to claim 7, wherein the compound is a di rect agonist of PDZ-GEF2.

10. The method according to claim 7, wherein the compound increases integrity of the cell-cell junction.

11. The method according to claim 7, wherein the agonist decreases permeability of an endothelial and/or epithelial wall.

12. The method according to claim 7, wherein the compound binds to PDZ-GEF2.

13. A method of selecting a compound capable of binding to PDZ-GEF2, the method comprising:
producing protein and/or fragments and/or peptides of 5 to 100 amino acids of a protein having an amino acid sequence corresponding to amino acid sequence 1-1601 of PDZ-GEF2 (SEQ ID NO:______),
contacting a plurality of candidate compounds to the protein and/or fragments and/or peptides of 5 to 100 amino acids of a protein having an amino acid sequence corresponding to amino acid sequence 1-1601 of PDZ-GEF2 (SEQ ID NO:______), and
selecting a compound that binds to the protein, and/or one or more fragments and/or one or more peptides,
contacting the compound with the PDZ-GEF2, and
determining whether the compound is capable of binding to PDZ-GEF2.

14. An agonist of PDZ-GEF2, the agonist selected by the method according to claim 7.

15. A medicament comprising the agonist of PDZ-GEF2 of claim 14.

16. A method of decreasing vascular leakage and/or for increasing epithelial integrity, the method comprising:
using the agonist of PDZ-GEF2 of claim 14 to decrease vascular leakage and/or increase epithelial integrity.

17. The medicament of claim 15 further comprising a suitable carrier.

18. A cell wherein PDZ-GEF2 is at least partially down-regulated, the cell characterized by immature cell-cell junctions and/or a decreased concentration of PDZ-GEF2 compared to a natural cell of the same kind.

19. A selection assay for an agonist of PDZ-GEF2 comprising using the cell of claim 18 in a selection assay for an agonist of PDZ-GEF2.

20. A selection assay for a compound improving the integrity of endothelial and/or epithelial cell-cell junctions, the selection assay comprising:
using the cell of claim 18 for a selection assay for a compound improving the integrity of endothelial and/or epithelial cell-cell junctions.
